(19) United States
(12) Patent Application Publication
(10) Pub. No.: US 2025/0018100 A1
(43) Pub. Date: Jan. 16, 2025

(54) PERITONEAL DIALYSIS SYSTEM HAVING A PATIENT LINE FILTER

(71) Applicants: BAXTER INTERNATIONAL INC., Deerfield, IL (US); BAXTER HEALTHCARE SA, Glattpark (Opfikon) (CH)

(72) Inventors: Steffen WAGNER, Deerfield, IL (US); Ralf FLIEG, Deerfield, IL (US); Reinhold BUCK, Deerfield, IL (US); Christof BECK, Deerfield, IL (US); Rainer BLICKLE, Deerfield, IL (US); Bernd KRAUSE, Deerfield, IL (US); Torsten KNOER, Deerfield, IL (US)

(21) Appl. No.: 18/710,712

(22) PCT Filed: Nov. 18, 2022
(86) PCT No.: PCT/US22/80157

§ 371 Date: May 16, 2024

Related U.S. Application Data
(60) Provisional application No. 63/290,855, filed on Dec. 17, 2021.

(51) Int. Cl. A61M 1/16; A61M 1/28

(52) U.S. Cl. CPC A61M 1/1631; A61M 1/288; A61M 2205/7509

(57) ABSTRACT

A peritoneal dialysis (“PD”) system (10) includes a PD machine (20); a patient line (50) extending from the PD machine (20); and a filter set (100) in fluid communication with the patient line (50), the filter set (100) including a filter membrane (120, such as a sterilizing grade or a bacteria reduction filter membrane) positioned and arranged such that fresh PD fluid flows through the filter membrane (120) into a filtered fluid compartment (106f), wherein the filtered fluid compartment (106f) includes an outlet (106t) to a port (106p), and wherein the port (106p) is in fluid communication with a used PD fluid tube (106u) positioned and arranged to carry used PD fluid past the filter membrane (120) without contacting the filter membrane (120). A method for priming filter set (100) is also disclosed.

### PRIORITY CLAIM

The present application claims priority to and the benefit of U.S. Provisional Application No. 63/290,855, filed on Dec. 17, 2021, the entire contents of which are hereby incorporated by reference.

### BACKGROUND

The present disclosure relates generally to medical fluid treatments and in particular to the filtering of treatment fluid during dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

PD fluid needs to be sterile or very near sterile because it is injected into the patient's peritoneal cavity, and is accordingly considered a drug. While bagged PD fluid is typically properly sterilized for treatment, PD fluid made online or PD machines or cyclers that employ disinfection may need additional sterilization.

There is accordingly a need for an effective, low cost way of providing additional sterilization to fresh PD fluid before it is delivered to a patient.

### SUMMARY

The present disclosure provides a peritoneal dialysis (“PD”) system having a PD machine or cycler that pumps fresh PD fluid through a patient line to a patient and removes used PD fluid from the patient via the patient line. The patient line may be reusable or disposable and in either case operates with and fluidly communicates with a filter set. If the patient line is reusable, the reusable patient line is connected to the filter set at the time of treatment. If the patient line is disposable, the filter set is merged into the disposable patient line in one embodiment. In either configuration a distal end of the filter set may be connected to the patient's transfer set, which in turn communicates fluidly with the patient's indwelling catheter.

The PD machine or cycler may include a durable PD fluid pump that pumps PD fluid through the pump itself without using a disposable component, or a disposable type PD fluid pump including a pump actuator that actuates a disposable, fluid-contacting pumping component, such as a peristaltic pump tube or a flexible pumping chamber. The PD machine or cycler also includes a plurality of valves, which may likewise be flow-through and durable without operating with a disposable component, or be disposable type valves having valve actuators that actuate a disposable, fluid-contacting valve component, such as a tube segment or a cassette-based valve seat.

The pumps and valves are under the automatic control of a control unit provided by the machine or cycler. In an embodiment, the valves include a fresh PD fluid valve that the control unit opens to allow the PD fluid pump to pump fresh PD fluid through a fresh PD fluid lumen of a dual lumen patient line to the patient. The valves also include a used PD fluid valve that the control unit opens to allow the PD fluid pump to pump used PD fluid from the patient through a used PD fluid lumen of the dual lumen patient line. It should be appreciated that while a single PD fluid pump may be used, dedicated fresh and used PD fluid pumps may be used alternatively. Also, a single PD fluid pump may include multiple pumping chambers for more continuous PD fluid flow.

The fresh and used PD fluid lumens may again be reusable or disposable. In the instance in which the fresh and used PD fluid lumens are reusable, the lumens terminate with a connector that connects to a lumen-side connector of the filter set, which may be sealed to (e.g., ultrasonically sealed, heat sealed or solvent bonded) or molded with a body of the filter set. The body is in turn sealed to (e.g., ultrasonically sealed, heat sealed or solvent bonded) or molded with a transfer set-side connector that either connects directly to a mating connector of the patient's transfer set or to a mating connector of a short tube placed between the body and the patient's transfer set. The transfer set-side connector may alternatively be placed at the end of a short tube that extends form the body. Here, the body provides (e.g., is molded with) a transfer set-side port to which the short tube extends into or over for welding to the port. The body, lumen-side connector, and transfer set-side connector or transfer set-side port may be referred to herein as a filter housing.

The lumen-side connector and the body form a fresh PD fluid passageway and a used PD fluid passageway. The fresh PD fluid passageway extends through a fresh PD fluid port in the lumen-side connector and towards a wall located within the body of the filter housing. The wall forces the fresh PD fluid to change directions and flow over the wall into an outer compartment, which resides over the outside of a flat sheet filter membrane. The fresh PD fluid is pressurized within the outer compartment. The pressurization forces the fresh PD fluid through the flat sheet filter membrane and into a filtered fluid compartment of the body, which is bounded primarily by the inner surface of the flat sheet filter membrane and a ribbed wall having a series of ribs that support the flat sheet filter membrane, especially while under a negative patient drain pressure.

The filter membrane may be a sterilizing grade or a bacteria reduction grade hydrophilic membrane, which may be formed with porous walls having a pore size of about 0.2 micron, through which the fresh PD fluid flows for further filtration. The flat sheet filter membrane is sized to provide the necessary filtration needed over multiple patient fills of a PD treatment.

Fresh and further filtered PD fluid flows in one embodiment from the filtered fluid compartment of the body, through an outlet provided in the ribbed wall and into a transfer set-side port, through the transfer set-side port, through the short tube of the filter set, and through the patient's transfer set, into the patient's peritoneal cavity. The short tube extends over the transfer set-side port where it is ultrasonically sealed, heat sealed or solvent bonded to the transfer set-side port. The transfer set-side port extends to an internal used PD fluid tube, which resides in one embodiment adjacent to the filtered fluid compartment. The used PD fluid tube may be molded with the primary portion of the body. The used PD fluid tube extends from the transfer set-side port to a used PD fluid port provided by the lumen-side connector, collectively forming the used PD fluid passageway. The used PD fluid port is in sealed fluid communication during operation to the used PD fluid lumen of the dual lumen patient line.

The used PD fluid tube enables used PD fluid to be pulled through the body of the filter housing without contacting and potentially clogging the filter membrane. The used PD fluid tube also provides a clear, straight path for the used PD fluid, which helps to mitigate against pressure losses due to the filter set. While it is fluidically possible for used PD fluid to flow through the outlet provided in the ribbed wall and into the filtered fluid compartment of the body, negative pressure is applied only from within the used PD fluid tube, so there is little incentive for used PD fluid to flow into the filtered fluid compartment. Likewise, while it is fluidically possible for fresh PD fluid to flow in reverse back up the used PD fluid tube, the change in direction required makes such a path much more tortuous than simply flowing through the transfer set-side port to the patient. Also, the used PD fluid tube and the used PD fluid lumen of the dual lumen patient tube are likely full of PD fluid during a patient fill, and the used PD fluid lumen is closed off at the PD machine or cycler, so there is little or no room for fresh PD fluid to enter the used PD fluid tube.

If needed, it is contemplated to cover the outlet provided in the ribbed wall with at least one hinged closure flap. The hinge or hinges may be a living hinge or hinges, wherein the at least one closure flap is molded with the ribbed wall along one or more side of the outlet. The at least one closure flap opens under positive pressure applied by the just-filtered fresh PD fluid, allowing the PD fluid to flow out of the outlet and into the transfer set-side port. The flow of used PD fluid across the at least one closure flap closes the flap so that used PD fluid cannot flow through the outlet provided in the ribbed wall and into the filtered fluid compartment. It is contemplated to form the at least one hinged closure flap so that the flap is biased closed when not under positive pressure from the fresh PD fluid. The at least one hinged closure flap provides one-way or check valve functionality without requiring a separate valve.

As mentioned above, the ribbed wall partially forming the filtered fluid compartment of the body is provided in one embodiment with a series of ribs that support the flat sheet filter membrane, especially when placed under negative fluid pressure. In this manner, the filter membrane may be as large as it needs to be to provide a desired filtration capacity. The ribbed wall is co-molded with the used PD fluid tube, the lumen-side connector and the transfer set-side connector in one embodiment. The flat sheet filter membrane is sealed in place via ultrasonic sealing, heat sealing or solvent bonding to an internal wall of the body that borders the ribbed wall.

The body of the filter housing includes a cap, which may be formed from the same material as the remainder of the body of the filter housing. The cap may be ultrasonically sealed, heat sealed or solvent bonded to outer portions of the sidewalls of the body. A tongue and groove fit may be provided between the cap and the sidewalls to center the cap for sealing. The cap forms the outside of the outer compartment into which fresh PD fluid flows before passing through the filter membrane.

The cap may be formed with one or more air vent. Each vent is covered on the inside of the cap with a hydrophobic membrane, which may be ultrasonically sealed, heat sealed or solvent bonded to the inside surface of the cap and around the at least one vent opening. The one or more vent and hydrophobic membrane allow air to be vented to atmosphere as the fresh PD fluid is pressurized within the outer compartment of the body prior to being filtered through the hydrophilic membrane, which may improve the performance of the membrane in addition to removing air from the filter set. In an embodiment, the cap is provided with one or more protective rib located adjacent to the one or more air vent. The one or more protective rib helps to prevent the one or more air vent from being covered by the patient, blanket, etc., while the patient sleeps during the PD treatment.

A gasket, such as a silicone or polyvinyl chloride (“PVC”) rubber gasket, may be fitted onto and/or into the fresh and used PD fluid ports of the lumen-side connector of the filter housing. The patient line connector may include fresh and used ports that extend into the fresh and used PD fluid ports of the lumen-side connector. The gasket provides port seals between the mated fresh and used PD fluid ports of the patient line connector and the lumen-side connector.

Used PD fluid removed through the patient's transfer set travels under negative pressure through the filter set via the used PD fluid tube (thus bypassing the filter membrane), through the used PD fluid lumen of the dual lumen patient line, and back to the machine or cycler. The machine or cycler pumps the used PD fluid under positive pressure to drain. The cycler includes a pressure sensor located along the used PD fluid side of its internal tubing, which measures the negative pressure applied by the PD fluid pump to the used PD fluid during a patient drain. That same pressure sensor may be used during a patient fill to measure the positive pumping pressure (which may, if not blocked by the at least one closure flap, be transmitted back through the used PD fluid tube of the filter set and used PD fluid lumen of the patient line to the pressure sensor), which is desirable because the measured pressure is of the fresh PD fluid downstream (after filtration) of the filter membrane. The measured pressure accordingly takes into account any pressure drop across the filter membrane, which more accurately reflects the pressure at which the PD fluid is being delivered to the patient.

In light of the disclosure set forth herein, and without limiting the disclosure in any way, in a first aspect of the present disclosure, which may be combined with any other aspect, or portion thereof, a peritoneal dialysis (“PD”) system includes a PD machine; a patient line extending from the PD machine; and a filter set in fluid communication with the patient line, the filter set including a filter membrane positioned and arranged such that fresh PD fluid flows through the filter membrane into a filtered fluid compartment, wherein the filtered fluid compartment includes an outlet to a port, and wherein the port is in fluid communication with a used PD fluid tube positioned and arranged to carry used PD fluid past the filter membrane without contacting the filter membrane.

In a second aspect of the present disclosure, which may be combined with any other aspect, or portion thereof, the patient line is a dual lumen patient line including a fresh PD fluid lumen and a used PD fluid lumen, the used PD fluid lumen placed in fluid communication with the used PD fluid tube.

In a third aspect of the present disclosure, which may be combined with any other aspect, or portion thereof, the filter set includes a fresh PD fluid port for fluid communication with the fresh PD fluid lumen and a used PD fluid port for fluid communication with the used PD fluid lumen.

In a fourth aspect of the present disclosure, which may be combined with any other aspect, or portion thereof, the used PD fluid tube is in fluid communication with the used PD fluid port.

In a fifth aspect of the present disclosure, which may be combined with any other aspect, or portion thereof, the port extends to used PD fluid tube.

In a sixth aspect of the present disclosure, which may be combined with any other aspect, or portion thereof, the outlet is releasably covered by at least one hinged closure flap.

In a seventh aspect of the present disclosure, which may be combined with any other aspect, or portion thereof, the PD system includes at least one rib located within the filtered fluid compartment for supporting the filter membrane.

In an eighth aspect of the present disclosure, which may be combined with any other aspect, or portion thereof, the filter membrane is a flat sheet filter membrane, and wherein the filter set includes an outer compartment located on an opposing side of the flat sheet filter membrane from the filtered fluid compartment.

In a ninth aspect of the present disclosure, which may be combined with any other aspect, or portion thereof, the filter set includes a fresh PD fluid port positioned and arranged to introduce fresh PD fluid to the outer compartment.

In a tenth aspect of the present disclosure, which may be combined with any other aspect, or portion thereof, the filter set includes a deflecting wall positioned and arranged to deflect fresh PD fluid from the fresh PD fluid port towards the outer compartment.

In an eleventh aspect of the present disclosure, which may be combined with any other aspect, or portion thereof, the filter set includes a cap cooperating with the filter membrane to form the outer compartment.

In a twelfth aspect of the present disclosure, which may be combined with any other aspect, or portion thereof, the cap includes at least one vent opening and at least one hydrophobic membrane sealingly covering the at least one vent opening.

In a thirteenth aspect of the present disclosure, which may be combined with any other aspect, or portion thereof, the filter set is configured to connect directly to a patient's transfer set, or wherein the filter set includes a flexible tube configured to connect to the patient's transfer set.

In a fourteenth aspect of the present disclosure, which may be combined with any other aspect, or portion thereof, the PD machine includes a pressure sensor positioned and arranged to sense the pressure of fresh PD fluid downstream from the filter membrane during a patient fill.

In a fifteenth aspect of the present disclosure, which may be combined with any other aspect, or portion thereof, the PD machine is configured to close a used PD fluid valve during a patient fill, urging the filtered fresh PD fluid to flow to the port instead of along the used PD fluid tube.

In a sixteenth aspect of the present disclosure, which may be combined with any other aspect, or portion thereof, the PD machine is configured to close a fresh PD fluid valve during a patient drain, urging used PD fluid to flow along the used PD fluid tube instead of into the filtered fluid compartment.

In a seventeenth aspect of the present disclosure, which may be combined with any other aspect, or portion thereof, the filter membrane is a sterilizing grade filter membrane or a bacteria reduction filter membrane.

In an eighteenth aspect of the present disclosure, which may be combined with any other aspect, or portion thereof, a filter set includes a port; a filtered fluid compartment including an outlet to the port; a filter membrane positioned and arranged such that fresh PD fluid flows through the filter membrane into the filtered fluid compartment; and a used PD fluid tube in fluid communication with the port, the used PD fluid tube positioned and arranged to carry used PD fluid past the filter membrane without contacting the filter membrane.

In a nineteenth aspect of the present disclosure, which may be combined with any other aspect, or portion thereof, a method is provided for priming a filter set connected to a dual lumen patient line, wherein during treatment a tube is located between the filter set and a patient's transfer set, the method including (i) delivering fresh peritoneal dialysis (“PD”) fluid through a fresh PD fluid lumen of the dual lumen patient line to the filter set; (ii) forcing the fresh PD fluid through a filter membrane of the filter set, so that the fresh PD fluid displaces air towards a used PD fluid lumen of the dual lumen patient line; and pulling used PD fluid from the patient, through the patient's transfer set, through the tube, through a used PD fluid portion of the filter set, and into the used PD fluid lumen of the dual lumen patient line.

In a twentieth aspect of the present disclosure, which may be combined with any other aspect, or portion thereof, pulling used PD fluid is provided as part of an initial patient drain.

In a twenty-first aspect of the present disclosure, which may be combined with any other aspect, or portion thereof, wherein between forcing the fresh PD fluid through the filter membrane and pulling used PD fluid from the patient, the patient is prompted to connect the tube to the patient's transfer set.

In a twenty-second aspect of the present disclosure, which may be combined with any other aspect, or portion thereof, wherein between forcing the fresh PD fluid through the filter membrane and pulling used PD fluid from the patient, the patient is prompted to open a clamp of the patient's transfer set.

In a twenty-third aspect of the present disclosure, which may be combined with any other aspect, or portion thereof, air is primed through at least one vent opening of the filter set while delivering fresh PD fluid through the fresh PD fluid lumen of the dual lumen patient line.

In a twenty-fourth aspect of the present disclosure, which may be combined with any other aspect, or portion thereof, a fresh PD fluid valve is open and a used PD fluid valve is closed while delivering fresh PD fluid through the fresh PD fluid lumen of the dual lumen patient line.

In a twenty-fifth aspect of the present disclosure, which may be combined with any other aspect, or portion thereof, a used PD fluid valve is open while forcing the fresh PD fluid through the filter membrane of the filter set.

In a twenty-sixth aspect of the present disclosure, which may be combined with any other aspect, or portion thereof, a used PD fluid valve is open while pulling used PD fluid from the patient.

In a twenty-seventh aspect of the present disclosure, which may be combined with any other aspect, or portion thereof, the method includes accumulating known volume pump strokes to control a volume pumped to force the fresh PD fluid through the filter membrane.

In a twenty-eighth aspect of the present disclosure, which may be combined with any other aspect, or portion thereof, the method includes sensing a pressure increase to transition from (i) delivering fresh PD fluid through the fresh PD fluid lumen of the dual lumen patient line to the filter set to (ii) forcing the fresh PD fluid through the filter membrane.

In a twenty-ninth aspect of the present disclosure, which may be combined with any other aspect, or portion thereof, the used PD fluid portion of the filter set includes a used PD fluid tube.

In a thirtieth aspect of the present disclosure, which may be combined with any other aspect, or portion thereof, the used PD fluid pulled from the patient is residual effluent from a previous treatment left for the purpose of priming the tube.

In a thirty-first aspect of the present disclosure, which may be combined with any other aspect, or portion thereof, a volume of the residual effluent is at least 50 ml.

In a thirty-second aspect of the present disclosure, which may be combined with any other aspect, or portion thereof, any of the features, functionality and alternatives described in connection with any one or more of FIGS. 1 to 7 may be combined with any of the features, functionality and alternatives described in connection with any other of FIGS. 1 to 7.

In light of the above aspects and the description herein, it is an advantage of the present disclosure to provide a filter set that operates with a dual lumen patient line.

It is another advantage of the present disclosure to provide a filter set that filters fresh PD fluid and allows used PD fluid to pass without contacting the filter membrane.

It is a further advantage of the present disclosure to provide a filter set that vents air from the fresh PD fluid before it is further filtered by the filter set.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of one embodiment for a peritoneal dialysis system having a filter set of the present disclosure.

FIG. 2 is a sectioned perspective view of one embodiment for a filter housing of the filter set of the present disclosure during a patient fill.

FIG. 3 is a perspective view of one embodiment for an optional lumen-side connector of the filter housing of the filter set of the present disclosure.

FIG. 4 is a perspective view highlighting a filtered fluid compartment and associated filter membrane support ribs, and a used PD fluid tube of one embodiment for a filter set of the present disclosure.

FIG. 5 is a sectioned perspective view of one embodiment for a filter housing of the filter set of the present disclosure during a patient drain.

FIG. 6 is a sectioned perspective view illustrating one embodiment for providing hinged closure flaps for sealing the used PD fluid tube of the filter housing of the filter set of the present disclosure.

FIG. 7 is a sectioned perspective view illustrating another embodiment for providing hinged closure flaps for sealing the used PD fluid tube of the filter housing of the filter set of the present disclosure.

### DETAILED DESCRIPTION

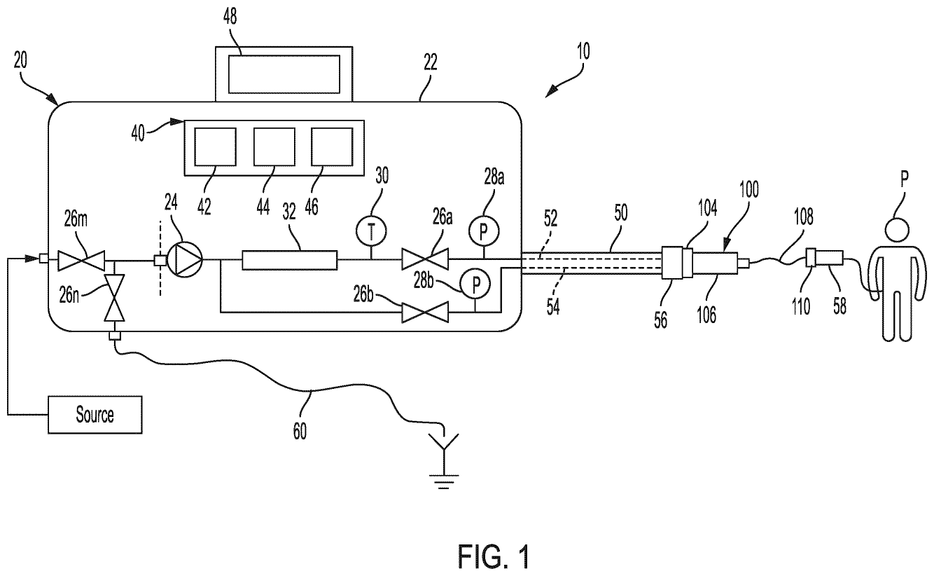

Referring now to the drawings and in particular to FIG. 1, a peritoneal dialysis (“PD”) system 10 is illustrated. PD system 10 includes a PD machine or cycler 20 that pumps fresh PD fluid through a patient line 50 to a patient P and removes used PD fluid from patient P via patient line 50. Patient line 50 may be reusable or disposable and in either case operates with and fluidly communicates with a filter set 100. If patient line 50 is reusable, the reusable patient line is connected to filter set 100 at the time of treatment. If patient line 50 is instead disposable, filter set 100 is merged into or formed with disposable patient line 50 in one embodiment. In either configuration, a distal end of filter set 100 may be connected to the patient's transfer set 58, which in turn communicates fluidly with the indwelling catheter of patient P.

PD machine or cycler 20 may include a housing 22 providing a durable PD fluid pump 24 that pumps PD fluid through the pump itself without using a disposable component. Examples of durable pumps that may be used for PD fluid pump 24 include piston pumps, gear pumps and centrifugal pumps. Certain durable pumps, such as piston pumps are inherently accurate, so that machine or cycler 20 does not require additional volumetric control components. Other durable pumps, such as gear pumps and centrifugal pumps may not be as accurate, such that machine or cycler 20 provides a volumetric control device such as one or more flowmeter (not illustrated).

Pump 24 may alternatively be a disposable type PD fluid pump, which includes a pump actuator that actuates a disposable, fluid-contacting pumping component, such as a peristaltic pump tube or a flexible pumping chamber. Examples of disposable PD fluid pumps that may be used for PD fluid pump 24 include rotary or linear peristaltic pump actuators that actuate tubing, pneumatic pump actuators that actuate cassette sheeting, electromechanical pump actuators that actuate cassette sheeting and platen pump actuators that actuate tubing. It should be appreciated that while a single PD fluid pump 24 may be used, dedicated fresh and used PD fluid pumps may be used alternatively. Also, single PD fluid pump 24 may include multiple pumping chambers for more continuous PD fluid flow.

PD machine or cycler 20 also includes a plurality of valves 26a, 26b, 26m, 26n which may likewise be flow-through and durable without operating with a disposable component, or be disposable type valves having valve actuators that actuate a disposable, fluid-contacting valve component, such as a tube segment or a cassette-based valve seat. Examples of durable valves that may be used for valves 26a, 26b, 26m, 26n include flow-through solenoid valves. Such valves may be two-way or three-way valves. Examples of disposable valves that may be used for valves 26a, 26b, 26m, 26n include solenoid pinch valves that pinch closed flexible tubing, pneumatic valve actuators that actuate cassette sheeting, and electromechanical valve actuators that actuate cassette sheeting.

Machine or cycler 20 likely includes many valves 26a to 26n. For ease of illustration, machine or cycler 20 is shown having a fresh PD fluid valve 26a that is controlled to open to allow PD fluid pump 24 to pump fresh PD fluid under positive pressure through a fresh PD fluid lumen 52 of dual lumen patient line 50 to patient P. The valves also include a used PD fluid valve 26b that is controlled to open to allow PD fluid pump 24 to pull used PD fluid from patient P under negative pressure through a used PD fluid lumen 54 of dual lumen patient line 50. The valves also include one or more supply valve 26m that is controlled to open to allow fresh PD fluid to be pulled from one or more fresh PD fluid source. The valves further include a drain valve 26n that is controlled to allow used PD fluid to be delivered to a house drain or drain container via a drain line 60.

Machine or cycler 20 in the illustrated embodiment also includes pressure sensors, such as pressure sensors 28a, 28b. Pressure sensor 28a is located just downstream from fresh PD fluid valve 26a, while pressure sensor 28b is located just upstream from used PD fluid valve 26. Pressure sensor 28a may accordingly sense the pressure in fresh PD fluid lumen 52 of dual lumen patient line 50 even if fresh PD fluid valve 26a is closed, while pressure sensor 28b may sense the pressure in used PD fluid lumen 54 of dual lumen patient line 50 even if used PD fluid valve 26b is closed. Additionally, pressure sensor 28a is positioned to sense the pressure of fresh PD fluid upstream from a filter membrane 120 discussed herein during a patient fill. Pressure sensor 28b perhaps more importantly is positioned to sense the pressure of fresh PD fluid downstream (after filtration) from filter membrane 120 during a patient fill. The measured pressure via pressure sensor 28b accordingly takes into account any pressure drop across filter membrane 120, which more accurately reflects the pressure at which fresh PD fluid is being delivered to patient P.

Pump 24 and valves 26a to 26n in the illustrated embodiment are under the automatic control of a control unit 40 provided by machine or cycler 20 of system 10, while pressure sensors 28a, 28b (and other sensors) output to control unit 40. Control unit 40 in the illustrated embodiment includes one or more processor 42, one or more memory 44 and a video controller 46. Control unit 40 receives, stores and processes signals or outputs from pressure sensors 28a, 28b, and other sensors provided by machine or cycler 20, such as one or more temperature sensor 30 and one or more conductivity sensor (not illustrated). Control unit 40 may use pressure feedback from one or more of pressure sensor 28a, 28b to control PD fluid pump 24 to pump dialysis fluid at a desired pressure or within a safe pressure limit (e.g., within 0.21 bar (three psig) of positive pressure to a patient's peritoneal cavity and −0.10 bar (−1.5 psig) of negative pressure from the patient's peritoneal cavity).

Control unit 40 uses temperature feedback from one or more temperature sensor 30 for example to control a heater 32, such as an inline heater to heat fresh PD fluid to a desired temperature, e.g., body temperature or 37° C. In one embodiment, heater 32 is used additionally to heat a disinfection fluid, such as fresh PD fluid, to disinfect PD fluid pump 24, valves 26a to 26n, heater 32 and all reusable fluid lines within machine or cycler 20 to ready the machine or cycler for a next treatment. The additional filtration discussed herein provides a layer of protection in addition to the heated fluid disinfection to ensure that the PD fluid is safe for delivery to patient P.

Video controller 46 of control unit 40 interfaces with a user interface 48 of machine or cycler 20, which may include a display screen operating with a touchscreen and/or one or more electromechanical button, such as a membrane switch. User interface 48 may also include one or more speaker for outputting alarms, alerts and/or voice guidance commands. User interface 48 may be provided with the machine or cycler 20 as illustrated in FIG. 1 and/or be a remote user interface operating with control unit 40. Control unit 40 may also include a transceiver (not illustrated) and a wired or wireless connection to a network, e.g., the internet, for sending treatment data to and receiving prescription instructions from a doctor's or clinician's server interfacing with a doctor's or clinician's computer.

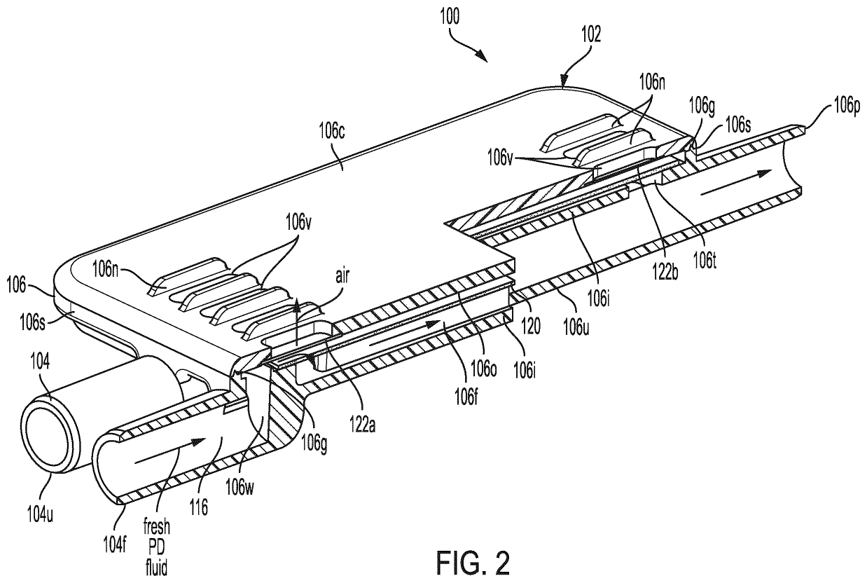

Referring to FIGS. 1 and 2, as mentioned above, fresh and used PD fluid lumens 52 and 54 of dual lumen patient line 50 may again be reusable or disposable. In the instance in which dual lumen patient line 50 is reusable, the lumens terminate with a connector 56 that connects to a lumen-side connector 104 of filter set 100, which may be sealed to (e.g., ultrasonically sealed, heat sealed or solvent bonded) or molded with a body 106 of the filter set. Body 106 in the illustrated embodiment is connected to a short, e.g., flexible, tube 108 that extends to a transfer set-side connector 110, which connects directly to a mating connector of the patient's transfer set 58. Short, e.g., flexible, tube 108 allows rigid lumen-side connector 104 and body 106 to be separated from rigid transfer set-side connector 110 to aid patient comfort. Forming body 106 to include transfer set-side connector 110, or attaching transfer set-side connector 110 to body 106, and then connecting those rigid structures to the patient's rigid transfer set 58 may lead to a combined rigid assembly that is uncomfortably tethered to patient P. The space provided by tube 108 separates body 106 from transfer set-side connector 110 so that only the rigid transfer set-side connector is mechanically connected to the patient's transfer set 58. In an alternative embodiment, however, transfer-side connector 110 may be formed with or attached to body 106.

Filter set 100 as packaged may be provided with removable caps (not illustrated and assuming dual lumen patient line 50 is reusable) on either end of filter set 100 after the set is sterilized, e.g., via gamma radiation, steam or ethylene oxide, to maintain sterility. To use filter set 100, the patient or user removes and discards the caps.

As illustrated in FIG. 2, lumen-side connector 104 may simply include ports, e.g., fresh and used PD fluid ports 104f and 104u, to which fresh and used PD fluid lumens 52 and 54 respectively extend over or into for sealing. If dual lumen patient line 50 is reusable, patient line connector 56 may include a releasable clamp that releasably clamps onto fresh and used PD fluid ports 104f and 104u, e.g., compressing a gasket interacting between patient line connector 56 and PD fluid ports 104f and 104u. If dual lumen patient line 50 is disposable, fresh and used PD fluid lumens 52 and 54 may be ultrasonically sealed, heat sealed or solvent bonding to fresh and used PD fluid ports 104f and 104u, respectively.

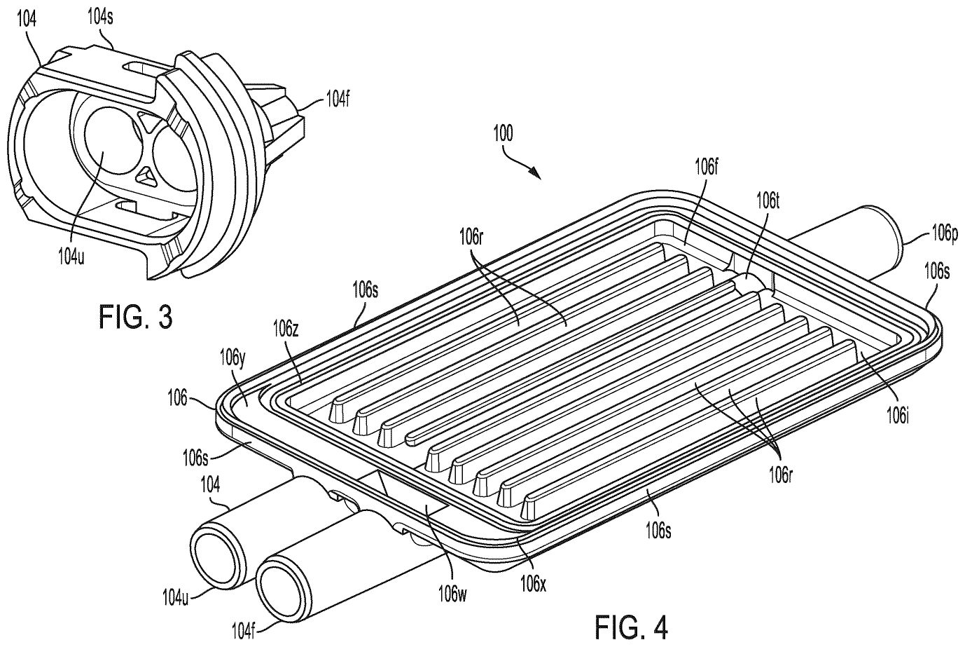

As illustrated in FIG. 3, in an alternative embodiment fresh and used ports 104f and 104u of lumen-side connector 104 are surrounded by a threaded shroud 104s, which may make a luer type connection with mating patient line connector 56. Here, patient line connector 56 is configured to thread onto threaded shroud 104s, causing the patient line connector 56 to compress a gasket (not illustrated) and seal mating fresh and used PD fluid ports of patient line connector 56 to fresh and used PD fluid ports 104f and 104u, respectively. The gasket may be fitted onto and/or into the fresh and used PD fluid ports 104f and 104u of the lumen-side connector 104. Patient line connector 56 may include fresh and used ports that extend into the fresh and used PD fluid ports 104f and 104u of lumen-side connector 104. The gasket in one embodiment provides port seals between the mated fresh and used PD fluid ports of patient line connector 56 and lumen-side connector 104.

Lumen-side connector 104 and body 106 may be referred to herein as a filter housing 102. Filter housing 102, transfer set-side connector 110, caps (not illustrated), and any other rigid or semi-rigid polymer associated with filter set 100 may be made of any one or more plastic, such as, polystyrene (“PS”), polycarbonate (“PC”), blends of polycarbonate and acrylonitrile-butadiene-styrene (“PC/ABS”), polyvinyl chloride (“PVC”), polyethylene (“PE”), polypropylene (“PP”), polyesters like polyethylene terephthalate (“PET”), or polyurethane (“PU”). Compressible gasket 112 may be formed from silicone rubber, PVC or other similar elastomeric material, such as styrene-ethylene-butylene-styrene (“SEBS”) or isoprene. Flexible tube 108 may be made of PVC or a non-PVC material, such as polybutadiene (“PBD”) or PP.

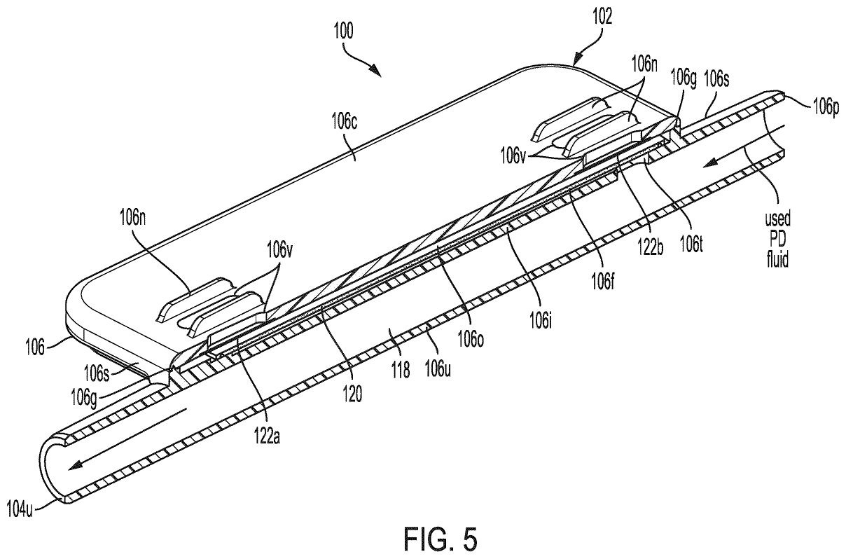

FIGS. 2 and 5 illustrate that a cap 106c is ultrasonically sealed, heat sealed or solvent bonded to the sidewalls 106s of body 106 to complete the body. Prior to sealing cap 106c to the sidewalls 106s of body 106, flat sheet filter membrane 120 is ultrasonically sealed, heat sealed or solvent bonded at its perimeter to inner portions of the sidewalls 106s of body 106. Flat sheet filter membrane 120 is made in one embodiment of a hydrophilic material that may have a pore size of about 0.2 micron through which fresh PD fluid flows for further filtration. Filter membrane 120 may be made of, for example, polysulfone or polyethersulfone blended with polyvinylpyrrolidone. Filter membrane 120 may be a sterilizing grade filter membrane or a bacteria reduction filter membrane.

FIGS. 2 and 5 further illustrate that cap 106c may be provided with vent openings 106v that allow air to be vented from the fresh PD fluid prior to being filtered through filter membrane 120. To maintain sterility within body 106, one or more hydrophobic membrane 122a, 122b, etc., is/are ultrasonically sealed, heat sealed or solvent bonded at its/their perimeter(s) to the inside surface of cap 106, so as to surround and cover its/their respective vent opening 106v. Hydrophobic membranes 122a, 122b, etc., may be made for example from polytetrafluoroethylene (“PTFE”). While multiple sets of vent openings 106v and corresponding hydrophobic membranes 122a, 122b are shown as being provided with cap 106c, only a single set of a vent opening and corresponding hydrophobic membrane may be provided with cap 106c.

One or more hydrophobic membrane 122a, 122b, etc., allows air to be vented to atmosphere as the fresh PD fluid is pressurized within an outer compartment 106o located beneath cap 106c prior to being filtered through the hydrophilic filter membrane 120, which may improve the performance of filter membrane 120 in addition to removing air from filter set 100. In the illustrated embodiment of FIGS. 2 and 5, cap 106c is provided with one or more protective rib 106n located adjacent to one or more air vent or vent opening 106v. One or more protective rib 106n helps to prevent one or more air vent 106v from being covered by the patient, blanket, etc., while the patient sleeps during the PD treatment.

Referring now to FIGS. 2, 4 and 5, lumen-side connector 104 and body 106 of filter housing 102 may be molded as a single structure except for cap 106c. Lumen-side connector 104 and body 106 form a fresh PD fluid passageway 116 and a used PD fluid passageway 118. As illustrated in FIG. 2, fresh PD fluid passageway 116 extends through fresh PD fluid port 104f in lumen-side connector 104 and towards a deflecting wall 106w located within body 106 of filter housing 102. Deflecting wall 106w forces the fresh PD fluid to change directions and flow along and over deflecting wall 106w into outer compartment 106o, which resides over the outside of flat sheet filter membrane 120. Outer compartment 106o is sized to distribute the fresh PD fluid across an upstream side of filter membrane 120 for even distribution through the porous membrane. Fresh PD fluid is pressurized within outer compartment 106o. The pressurization forces the fresh PD fluid through flat sheet filter membrane 120 and into a filtered fluid compartment 106f of body 106, which is bounded primarily by the underside surface of flat sheet filter membrane 120 and a ribbed wall 106i having a series of ribs 106r that support flat sheet filter membrane 120, especially while under a negative patient drain pressure.

FIG. 2 further illustrates that fresh and further filtered PD fluid flows in one embodiment from filtered fluid compartment 106f of body 106, through an outlet 106t provided in ribbed wall 106i and into a transfer set-side port 106p, through the transfer set-side port 106p, through short tube 108 of filter set 100, through the patient's transfer set 58, and into the peritoneal cavity of patient P. Transfer set-side port 106p extends from a used PD fluid tube 106u discussed in detail below. Short tube 108 (FIG. 1) extends over (or alternatively into) transfer set-side port 106p, where it is ultrasonically sealed, heat sealed or solvent bonded to the transfer set-side port 106p.

FIG. 4 illustrates ribbed wall 106i and sidewalls 106s of body 106 in more detail. Ribbed wall 106i and sidewalls 106s define filtered fluid compartment 106f. Ribbed wall 106i and sidewalls 106s are molded as one piece in the illustrated embodiment along with fresh and used PD fluid ports 104f, 104u of lumen-side connector 104, transfer set-side port 106p, series of ribs 106r, and used PD fluid tube 106u. Sidewalls 106s in the illustrated embodiment include or define a continuous centering sealing rib 106x which accepts a mating groove 106g (FIG. 2) formed along the underside perimeter of cap 106c. The fit of sealing rib 106x to groove 106g ensures that cap 106c is located properly for ultrasonic sealing, heat sealing or solvent bonding to sidewalls 106s.

FIG. 4 also illustrates that an internal wall 106y extends from sidewalls 106s inwardly to where ribbed wall 106i drops down from internal wall 106y to form filtered fluid compartment 106f. Internal wall 106y is similarly provided with a continuous (or beaded) ring of material 106z, which marks the placement position for flat sheet filter membrane 120. Ring of material 106z may help to center the placement flat sheet filter membrane 120 and/or provide additional material to help with the ultrasonic sealing, heat sealing or solvent bonding of filter membrane 120 to internal wall 106y.

The series of ribs 106r formed with ribbed wall 106i support flat sheet filter membrane 120 and allow the membrane to be as large as needed to final filter fresh PD fluid over multiple patient fills of a PD treatment. The series of ribs 106r support flat sheet filter membrane 120 especially under negative fluid pressure applied through used PD fluid port 104u of lumen-side connector 104. The series of ribs 106r also allow for the flow of filtered fresh PD fluid from filtered fluid compartment 106f to the outlet 106t.

FIGS. 2 and 4 in combination show how fresh PD fluid traveling through fresh PD fluid port 104f contacts deflecting wall 106w and flows upwardly into outer compartment 106o located beneath cap 106c. Fresh PD fluid disperses over flat sheet filter membrane 120 and is then pressurized through the porous wall of the filter membrane into filtered fluid compartment 106f, flowing along and around series of ribs 106r until exiting through outlet 106t. The exiting fresh PD fluid flows through transfer set-side port 106p to the patient.

FIG. 5 illustrates that transfer set-side port 106p extends to used PD fluid tube 106u, which resides in one embodiment adjacent to filtered fluid compartment 106f. Used PD fluid tube 106u may be molded with the primary portion of body 106. Used PD fluid tube 106u in the illustrated embodiment extends from transfer set-side port 106p to used PD fluid port 104u of lumen-side connector 104, collectively forming the used PD fluid passageway 118. Used PD fluid port 104u is in sealed fluid communication during operation to used PD fluid lumen 54 of dual lumen patient line 50 as discussed above.

Used PD fluid tube 106u enables used PD fluid to be pulled through body 106 of filter housing 102 without contacting and potentially clogging filter membrane 120. Used PD fluid tube 106u also provides a clear, straight path for the used PD fluid, e.g., having an inner diameter of 2 to 8 millimeters (“mm”), such as 4 mm, which helps to mitigate against pressure losses due to filter set 100.

While it is fluidically possible for used PD fluid to flow through outlet 106t provided in ribbed wall 106i and into the filtered fluid compartment 106f of body 106, negative pressure is applied only from within used PD fluid tube 106u, so there is little incentive for used PD fluid to flow into filtered fluid compartment 106f. Also, control unit 40 of PD machine 20 is configured to close fresh PD fluid valve 26a during a patient drain, urging used PD fluid to flow along the used PD fluid tube 106u instead of into the filtered fluid compartment 106f. Likewise, while it is fluidically possible during a patient fill for fresh PD fluid to flow in reverse back up used PD fluid tube 106u, the change in direction required makes such a path much more tortuous than simply flowing through transfer set-side port 106p to patient P. Also, used PD fluid tube 106u and used PD fluid lumen 54 of dual lumen patient tube 50 are likely full of fresh and/or used PD fluid during a patient fill, and used PD fluid lumen 54 is closed off via used PD fluid valve 26b at PD machine or cycler 20, so there is little or no room for fresh PD fluid to enter used PD fluid tube 106u.

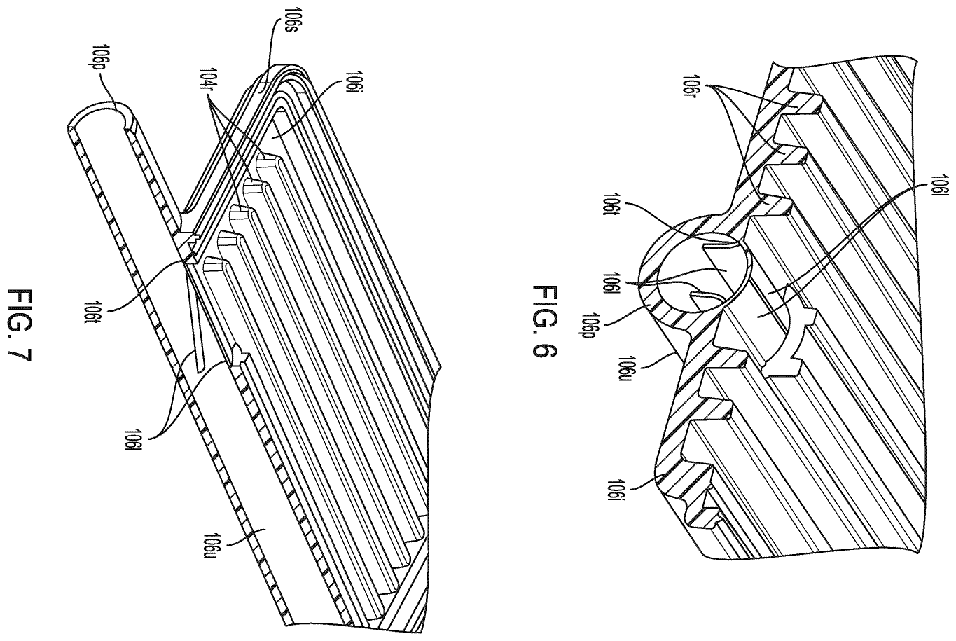

Referring now to FIGS. 6 and 7, different views of the outlet 106t from filtered fluid compartment 106f into transfer set-side port 106p are illustrated. Here, if needed, it is contemplated to cover outlet 106t provided in ribbed wall 106i with at least one hinged closure flap 1061. In FIG. 6, two closure flaps 1061 provided at the top of used PD fluid tube 106u and at outlet 106t hinge along longitudinal hinges. In FIG. 7, one or more closure flap 1061 provided at the top of used PD fluid tube 106u and at outlet 106t hinge(s) along at least one circumferential hinge. The hinge or hinges may be a living hinge or hinges, wherein at least one flap 1061 is molded with ribbed wall 106i along one or more side of outlet 106t. During a patient fill, At least one closure flap 1061 opens under positive pressure applied by the just-filtered fresh PD fluid, allowing the fresh PD fluid to flow out of outlet 106t and into transfer set-side port 106p. During a patient drain, the flow of used PD fluid across at least one closure flap 1061 closes the flap so that used PD fluid cannot flow through outlet 106t. It is contemplated to form the at least one hinged closure flap 1061 so that the flap is biased closed when not under positive pressure from the fresh PD fluid. At least one hinged closure flap 1061 accordingly provides one-way or check valve functionality without requiring a separate valve.

Regarding the priming of filter set 100 for treatment, fresh PD fluid lumen 52 of patient line 50 and filter set 100 may or may not be primed with fresh PD fluid before short tube 108 is connected to the patient's transfer set 58. If primed, user interface 48 may audibly, visually or audiovisually prompt patient P to clip patient line connector 56 and/or filter set 100 into a clip provided by housing 22 of PD machine or cycler 20. Short tube 108 may initially be fitted with a cap (not illustrated), so that when patient line connector 56 or filter set 100 is clipped to housing 22, short tube 108 hangs off of filter set 100 and is closed to the environment via the cap. Control unit 40 then causes PD fluid pump 24, with fresh PD fluid valve 26a open and used PD fluid valve 26b closed, to prime fresh PD fluid lumen 52 with fresh PD fluid up to filter membrane 120. Here, air is forced out vent openings 106v.

Once fresh PD fluid lumen 52 is primed fully, pressure sensors 28a and 28b sense a pressure increase because fresh PD fluid has nowhere to go with used PD fluid valve 26b closed. Upon seeing the pressure increase, with filter membrane 120 now fully wetted, control unit 40 then causes used PD fluid valve 26b to open, allowing PD fluid pump 24 to push fresh PD fluid through hydrophilic filter membrane 120 into filtered fluid compartment 106f, which pushes air through the inner compartment, into and through used PD fluid passageway 118 of used PD fluid tube 106u, and into a portion of used PD fluid lumen 54. Air is accordingly pushed up the used PD fluid lumen 54 towards system drain. Here, control unit 40 may be programed to know and actuate a number of known volume strokes of PD fluid pump 24 needed to adequately prime filtered fluid compartment 106f, used PD fluid passageway 118 of used PD fluid tube 106u, and a desired portion of used PD fluid lumen 54. At this point, body 106 of filter set 100 is fully primed. It should be appreciated that filter set 100 does not have to be clamped to housing 22 for the above priming of the body 106 of filter set 100 to be performed, however, doing so may help to prevent dual lumen patient line 50 from kinking during such priming.

User interface 48 of PD machine or cycler 20 then audibly, visually or audiovisually prompts patient P to remove filter set 100 from the clip at housing 22, to remove the cap from short tube 108, to connect short tube 108 to the patient's transfer set 58, and to open the clamp of the patient's transfer set 58. Control unit 40 then in an embodiment, with used PD fluid valve 26b open and fresh PD fluid valve 26a open or closed (likely closed), causes PD fluid pump 24 to pull used PD fluid from the patient to prime short tube 108, here pulling air from the short tube, through used PD fluid passageway 118 of used PD fluid tube 106u, up used PD fluid lumen 54 of dual lumen patient line 50, and towards the drain of PD machine or cycler 20. Such pulling of used PD fluid may be part of an initial drain of the patient. The amount of used PD fluid removed from the patient is accordingly counted at control unit 40 (e.g., by accumulating known volume strokes of PD fluid pump 24) as part of the treatment's initial drain volume in one embodiment.

If a patient fill is instead the first action to be taken after priming fresh PD fluid lumen 52 and the body 106 of filter set 100, control unit 40 may or may not pull effluent from the patient to fully prime short tube 108 prior to starting the initial patient fill. That is, it is contemplated to allow the small amount of air residing within short tube 108 to be pushed back to the patient. If however, control unit 40 does pull an initial amount of effluent from the patient to prime short tube 108, control unit 40 may count whatever amount of effluent is pulled from the patient (e.g., by accumulating known volume strokes of PD fluid pump 24) as part of a subsequent initial drain.

In an alternative embodiment, filter set 100 is not clipped at housing 22 and short tube 108 is initially connected to the patient's transfer set 58. User interface 48 here audibly, visually or audiovisually counsels patient P to leave the clamp of the patient's transfer set 58 closed until instructed to open the clamp. The procedure described above is then performed, wherein here the patient's transfer set clamp is performing the function of the cap at the end of short tube 108 in the above example. With the patient's transfer set clamp closed, control unit 40 causes fresh PD fluid to be primed through fresh PD fluid lumen 52, body 106 of filter set, and a portion of used PD fluid lumen 54 using PD fluid pump 24, while sequencing valves 26a and 26b as discussed above.

User interface 48 then prompts patient P to open the clamp of the patient's transfer set 58 and to press a confirm button at user interface 48 in one embodiment. Upon the confirm button being pressed, control unit 40 then sequences valves 26a and 26b and actuates pump 24 as discussed above to pull used PD fluid from the peritoneal cavity of patient P to prime short tube 108 and used PD fluid passageway 118 of used PD fluid tube 106u with patient effluent. The effluent priming of short tube 108 may again be part of an initial patient drain.

The pulling of used PD fluid from the patient to prime short tube 108 assumes that there is used PD fluid to remove from the patient at the start of treatment. This is true in many instances in which the patient is full of used PD fluid at the beginning of treatment from a previous treatment's last fill or from a midday exchange. In some instances, however, the patient is dry at the beginning of treatment. It is contemplated that control unit 40 of PD machine or cycler 20, which may be dedicated at a given time to a single patient, knows the patient's treatment schedule, and thus knows when the patient will begin a next treatment in a dry state with no or very little used PD fluid. It is contemplated here that control unit 40, instead of attempting to completely drain the patient in a final drain of a previous treatment, causes a residual amount of effluent to remain within the peritoneal cavity of the patient after treatment. The residual amount may for example be 50 milliliters (“ml”) or more as needed to ensure that the patient's indwelling PD catheter can access the residual effluent. The residual amount should be enough to prime any air at least through the proximal end of short tube 108 at the junction of filter set 100.

The above-described priming procedure is advantageous for a number of reasons. First, a step of having the patient clip patient line connector 56 into a clip provided by housing 22 of PD machine or cycler 20 may be eliminated. Also, the need for patient line connector 56 to be fitted with a vented cap and/or for housing 22 of PD machine or cycler 20 to have a sensor for detecting when fresh PD fluid has reached patient line connector 56 may be eliminated. Both savings reduce cost and complexity. Second, after treatment, the patient disconnects transfer set-side connector 110 from the patient's transfer set 58 and then seals transfer set 58 with a cap (not illustrated) having a disinfectant, such as iodine, to help prevent peritonitis due for example to patient touch contamination. The cap is then removed and replaced with a new transfer set-side connector 110 of a new filter set 100 at the beginning of a next treatment. Residual disinfectant, e.g., residual iodine, remains however. The priming method disclosed herein carries the residual disinfectant away into used PD fluid lumen 54 of dual lumen patient line 50 under negative pressure instead of delivering the residual disinfectant to the patient. Doing so may prevent health issues, especially for sensitive patients.

It should be understood that various changes and modifications to the presently preferred embodiments described herein will be apparent to those skilled in the art. It is therefore intended that any or all of such changes and modifications may be covered by the appended claims. For example, dual lumen patient line 50 could alternatively be a single lumen patient line, wherein filter set 100 includes check valves, e.g., provided within fresh and used PD fluid ports 104f, 104u of lumen-side connector 104 for directing fresh and used PD fluid to desired locations within the set.

1. A peritoneal dialysis (“PD”) system (10) comprising:
a PD machine (20);
a patient line (50) extending from the PD machine (20); and
a filter set (100) in fluid communication with the patient line (50), the filter set (100) including a filter membrane (120) positioned and arranged such that fresh PD fluid flows through the filter membrane (120) into a filtered fluid compartment (106f), wherein the filtered fluid compartment (106f) includes an outlet (106t) to a port (106p), and wherein the port (106p) is in fluid communication with a used PD fluid tube (106u) positioned and arranged to carry used PD fluid past the filter membrane (120) without contacting the filter membrane (120).

2. The PD system (10) according to claim 1, wherein the patient line (50) is a dual lumen patient line including a fresh PD fluid lumen (52) and a used PD fluid lumen (54), the used PD fluid lumen (54) placed in fluid communication with the used PD fluid tube (106u).

3. The PD system (10) according to claim 2, wherein the filter set (100) includes a fresh PD fluid port (104f) for fluid communication with the fresh PD fluid lumen (52) and a used PD fluid port (104u) for fluid communication with the used PD fluid lumen (54).

4. The PD system (10) according to claim 3, wherein the used PD fluid tube (106u) is in fluid communication with the used PD fluid port (104u).

5. The PD system (10) according to claim 1, wherein the port (106p) extends from the used PD fluid tube (106u).

6. The PD system (10) according to claim 1, wherein the outlet (106t) is releasably covered by at least one hinged closure flap (1061).

7. The PD system (10) according to claim 1, which includes at least one rib (106r) located within the filtered fluid compartment (106f) for supporting the filter membrane (120).

8. The PD system (10) according to claim 1, wherein the filter membrane (120) is a flat sheet filter membrane, and wherein the filter set (100) includes an outer compartment (1060) located on an opposing side of the flat sheet filter membrane from the filtered fluid compartment (106f).

9. The PD system (10) according claim 8, wherein the filter set (100) includes a fresh PD fluid port (104f) positioned and arranged to introduce fresh PD fluid to the outer compartment (1060).

10. The PD system (10) of claim 9, wherein the filter set (100) includes a deflecting wall (106w) positioned and arranged to deflect fresh PD fluid from the fresh PD fluid port (104f) towards the outer compartment (1060).

11. The PD system (10) of claim 8, wherein the filter set (100) includes a cap (106c) cooperating with the filter membrane (120) to form the outer compartment (1060).

12. The PD system (10) of claim 11, wherein the cap (106c) includes at least one vent opening (106v) and at least one hydrophobic membrane (122a, 122b) sealingly covering the at least one vent opening (106v).

13. The PD system (10) according to claim 1, wherein the filter set (100) is configured to connect directly to a patient's transfer set, or wherein the filter set (100) includes a flexible tube (108) configured to connect to the patient's transfer set.

14. The PD system (10) according to claim 1, wherein the PD machine (20) includes a pressure sensor (28b) positioned and arranged to sense pressure of fresh PD fluid downstream from the filter membrane (120) during a patient fill.

15. The PD system (10) according to claim 1, wherein the PD machine (20) is configured to close a used PD fluid valve (26b) during a patient fill, urging filtered fresh PD fluid to flow to the port (106p) instead of along the used PD fluid tube (106u).

16. The PD system (10) according to claim 1, wherein the PD machine (20) is configured to close a fresh PD fluid valve (26a) during a patient drain, urging the used PD fluid to flow along the used PD fluid tube (106u) instead of into the filtered fluid compartment (106f).

17. The PD system (10) according to claim 1, wherein the filter membrane (120) is a sterilizing grade filter membrane or a bacteria reduction filter membrane.

18. A filter set (100) comprising:
a port (106p);
a filtered fluid compartment (106f) including an outlet (106t) to the port (106p);
a filter membrane (120) positioned and arranged such that fresh PD fluid flows through the filter membrane (120) into the filtered fluid compartment (106f); and
a used PD fluid tube (106u) in fluid communication with the port (106p), the used PD fluid tube (106u) positioned and arranged to carry used PD fluid past the filter membrane (120) without contacting the filter membrane (120).

19. A method for priming a filter set (100) connected to a dual lumen patient line (50), wherein during treatment a tube (108) is located between the filter set (100) and a patient's transfer set (58), the method comprising:
delivering fresh peritoneal dialysis (“PD”) fluid through a fresh PD fluid lumen (52) of the dual lumen patient line (50) to the filter set (100);
forcing fresh PD fluid through a filter membrane (120) of the filter set (100), so that the fresh PD fluid displaces air towards a used PD fluid lumen (54) of the dual lumen patient line (50); and
pulling used PD fluid from a patient, through the patient's transfer set (58), through the tube (108), through a used PD fluid portion of the filter set (100), and into the used PD fluid lumen (54) of the dual lumen patient line (50).
